(19) United States
(12) Patent Application Publication
(10) Pub. No.: US 2007/0099879 A1
(43) Pub. Date: May 3, 2007

(54) METHOD OF USING CALCITRIOL FOR TREATING INTRAOCULAR DISEASES ASSOCIATED WITH ANGIOGENESIS

(75) Inventors: Nader SHEIBANI, Madison, WI (US); Daniel M. ALBERT, Madison, WI (US)

Correspondence Address: GODFREY & KAHN, S.C., 780 N. WATER STREET, MILWAUKEE, WI, 53202, US

(73) Assignee: Wisconsin Alumni Research Foundation (WARF)

(21) Appl. No.: 11/552,439

(22) Filed: Oct. 24, 2006

Related U.S. Application Data
(60) Provisional application No. 60/731,684, filed on Oct. 31, 2005.

(51) Int. Cl. A61K 31/59

(52) U.S. Cl. 514/167

(57) ABSTRACT

The present invention provides a method of treating pathologies resulting from neovascular growth in the eye such as those manifested as retinopathy of prematurity, diabetic retinopathy and macular degeneration. The invention comprises the administration of an effective amount of calcitriol that is administered at doses less than toxicity and results in a significant reduction in the formation of neo-vascular growth. The invention can be used to treat existing diseases or prophylactically to treat those at risk.

### CROSS-REFERENCE TO RELATED APPLICATIONS

This application claims the benefit of U.S. Provisional application 60/731,684, filed Oct. 31, 2005, incorporated herein be reference in its entirety.

### STATEMENT AS TO RIGHTS TO INVENTIONS MADE UNDER FEDERALLY SPONSORED RESEARCH OR DEVELOPMENT

This Work was supported in part by a grant from the National Institutes of Health Grant EY013700, DK67120 and EY001917. The Government of the United States of America may have certain rights in this invention.

### FIELD OF THE INVENTION

This invention is generally directed to methods of treating intraocular diseases associated with progressive angiogenesis. More particularly the invention recites the use of calcitriol to prevent or inhibit neovascular growth in the eye.

### BACKGROUND OF THE INVENTION

Angiogenesis, the process of the formation of new blood vessels from pre-existing capillaries, is tightly regulated and normally does not occur except during development, wound healing, and the formation of the corpus luteum during the female reproductive cycle. This strict regulation is manifested by a balanced production of positive and negative factors, which keep angiogenesis in check. However, this balance becomes abrogated under various pathological conditions, such as cancer, diabetes, age-related macular degeneration and retinopathy of prematurity (ROP), resulting in the growth of new blood vessels. It is now well accepted that the progressive growth and metastasis of many solid tumors and loss of vision with diabetes are dependent on the growth of new blood vessels. Therefore, there is great interest in the development and identification of agents that can inhibit angiogenesis as a means of treating a variety of diseases with a neovascular or angiogenic component.

Vascular diseases of the eye comprise a major cause of blindness and have only imperfect methods of treatment. These diseases include various retinopathies and macular degeneration. Retinopathy frequently results in blindness or severely limited vision due to unorganized growth and/or damage to retinal blood vessels. There are two major types of retinopathy: diabetic retinopathy and retinopathy of prematurity. Diabetic retinopathy affects nearly 80% of all diabetics who have had diabetes for more than 15 years. Retinopathy of prematurity is thought to result from oxygen toxicity, with about 15,000 premature infants a year being diagnosed with ROP in the United States alone. Macular degeneration results from the neovascular growth of the choroid vessel underneath the macula. There are two types of macular degeneration: dry and wet. While wet macular degeneration only comprises 15% of all macular degeneration, nearly all wet macular degeneration leads to blindness. In addition, wet macular degeneration nearly always results from dry macular degeneration. Once one eye is affected by wet macular degeneration, the condition almost always affects the other eye.

Though these diseases have different etiologies, they all result in damage to the normal ocular vasculature and result in abnormal growth of new vessels. While there is no cure for neovascular ocular diseases, there are three generally accepted treatments: laser ablation of various regions of the retina, thereby decreasing retinal oxygen consumption and hopefully, concomitant neovascular growth; vitrectomy or removal of the cloudy vitreous humor and its replacement with a saline solution; and administration of antioxidant vitamins E and C. In addition there has been some recent interest in the use of the synthetic vitamin B1, benfotiamine. In retinopathy of prematurity cryotherapy, which destroys the fringe of the retina through freezing, is the only treatment so far that has been proven to provide substantial benefit to the eye.

There are several animal models developed for studying retinal neovascular growth. They include the induction of diabetes in genetically predisposed rodents to induce ocular neovascularization. However, the models are limited in their predictive values to a diabetic model. By far one of the most accepted models of vasculopathies of the eye is the Oxygen-Induced IschemicRetinopathy (OIR) model described by Smith et al. (L E Smith, E Wesolowski, A McLellan, SK Kostyk, R D'Amato, R Sullivan, and PA D'Amore (1994) Oxygen-induced retinopathy in the mouse Invest. Ophthalmol. Vis. Sci. 35: 101-111). The use of the OIR model has allowed the investigation of neovascular growth mimicking both retinopathy of prematurity and proliferative diabetic retinopathy and also provided an overall model for assessing the physiological effects of various compounds, such as, growth factors, cytokines and drugs on inducible angiogenesis.

The anti-tumor activity of vitamin D compounds has been demonstrated in preclinical and/or clinical tests against a variety of cancers, including retinoblastoma. However, the molecular and cellular mechanisms responsible for tumor growth inhibition have not been identified. The reduced vascularity observed in many tumors treated with vitamin D compounds suggests tumor vasculature may be a target. The in vitro and in vivo studies have demonstrated that calcitriol can directly affect endothelial cell (EC) activity and impact their proliferation and sprouting (10-12). However, the effects of calcitriol on retinal vascularization have not been previously addressed. In addition, a cytotoxic effect of calcitriol treatment includes elevation of serum calcium levels, which can result in hardening of soft tissues and weight loss.

The effect of calcitriol on neovascular growth has not indicated any clear effect. Further, past studies examining the effect of calcitriol on retinoblastoma (see, Albert et al. Invest Ophthalmol Vis Sci. 1992 July;33(8):2354-64) have previously given no indications of its efficacy for the use of neovascular growth in the eye. In investigations on retinoblastomas, vitamin D given to mice at doses of 0.2 μg/d to 0.025 μg/d showed an inverse relationship between the amount of calcitriol and the degree of involvement of retinoblastoma throughout the eye. However, even those animals given a dose of 0.05 μg/d experienced weight loss and an increase in serum calcium consistent with vitamin D toxicity while experiencing only a 50% decrease in retinal involvement compared to controls. If calcitriol does exert an anti-neoplastic effect through anti-angiogenic mechanisms, its incomplete effect at doses bordering toxicity severely limits its therapeutic value.

Presently, pathologic conditions of the eye that are manifested by angiogenesis and neovascular growth of the retina have no cure. Further, methods of treating such pathologic conditions require invasive treatment and result in significant loss of vision. Non-invasive methods of treatment are experimental and have not been shown to substantially reduce the risk of blindness or loss of sight. Thus, there is a need for more effective methods of treatment that reduce and/or inhibit neovascular growth in the eye without requiring invasive techniques that also result in irreparable damage to the eye.

A treatment for neovascular growth and, in particular retinopathy, that did not require invasive surgery and was as suitable for infants as well as adults would greatly increase the treatment options and improve the prognosis for sufferers of various retinopathies. While understanding how vitamin D compounds inhibit angiogenesis and development of analogues that retain antiangiogenic activity but have no calcemic activity would provide an ideal treatment, using the tools currently available and understanding their action better may provide a more quickly obtainable treatment.

### SUMMARY OF THE INVENTION

Recently, 19-nor analogs of vitamin D have been synthesized that appear to separate the effects of the hormonal form of vitamin D on calcium homeostasis and cell growth. The inventors, in studying the effects of the 19-nor analogs on the OIR model of neovascular growth, used calcitriol as one control in their experiments. The effects of vitamin D compounds on the neovascular growth of mice during OIR were evaluated. The inventors unexpectedly found that, while the 19-nor compounds showed little effect, calcitriol had a significant effect on limiting neovascular growth compared to control animals. Thus, the inventors have shown that calcitriol is a potent inhibitor of angiogenesis and, in particular, neovascular growth in the retina. As disclosed herein, calcitriol is a potent inhibitor of neovascular growth in the eye in vivo, providing a valuable therapeutic treatment for ocular conditions manifested by neovascular growth such as diabetic retinopathy, retinopathy of prematurity and macular degeneration that has heretofore not existed. This invention provides a method of treatment for diseases resulting from ocular angiogenesis and neovascular growth comprising administration of an effective amount of calcitriol.

Accordingly, this invention provides a method of treating pathological conditions resulting from ocular angiogenesis and neovascular growth of the eye comprising administration of an effective amount of calcitriol.

In one exemplary embodiment, this invention provides a method of treating neovascular growth in the eye in a subject in need thereof comprising administering an effective amount of calcitriol having a structure represented by Formula I or a salt or prodrug thereof wherein neovascular growth is decreased in the eye.

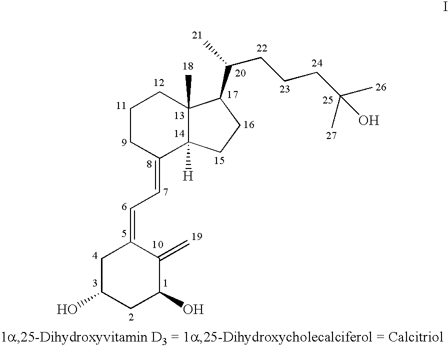

In another exemplary embodiment, this invention provides methods for using a composition suitable for treating angiogenesis and/or neovascular growth in the eye. The composition comprises: a first ingredient which inhibits angiogenesis comprising the compound, prodrug or salt of Formula I; and a second ingredient which comprises an acceptable carrier. In one exemplary embodiment, the acceptable carrier is a pharmaceutically acceptable carrier. Preferably, in the composition, the first ingredient is calcitriol.

In exemplary embodiments, the invention includes a method of treating non-neoplastic neovascular growth, such as, for example, various retinopathies of the eye and dermatological vasculopathies, such as, for example, vascular birthmarks, to a subject in need thereof comprising administering an effective amount of calcitriol having a structure represented by Formula I or a salt or prodrug thereof wherein the non-neoplastic neovascular growth is decreased.

In various exemplary embodiments, the neovascular forming condition to be treated is diabetic retinopathy, retinopathy of prematurity, hypertensive retinopathy or macular degeneration.

In yet another exemplary embodiment, this invention provides a method of inhibiting neovascular growth, comprising administering an amount of calcitriol having a structure represented by Formula I or a salt or prodrug thereof wherein neovascular growth is inhibited.

In various exemplary embodiments, the neovascular forming conditions to be inhibited are dermatological vasculopathies, diabetic retinopathy, retinopathy of prematurity, hypertensive retinopathy and macular degeneration.

In another exemplary embodiment, this invention provides methods for using a composition suitable for inhibiting angiogenesis and/or neovascular growth. The composition comprises: a first ingredient which inhibits angiogenesis comprising the compound, prodrug or salt of Formula I; and a second ingredient which comprises an acceptable carrier. In one exemplary embodiment, the acceptable carrier is a pharmaceutically acceptable carrier. Preferably, in the composition, the first ingredient is calcitriol.

In sum, the present invention represents new methods of treating various diseases and/or pathological conditions resulting from angiogenesis and neovascular growth. These and other features and advantages of various exemplary embodiments of the methods according to this invention are described in, or are apparent from, the following detailed description of various exemplary embodiments of the methods according to this invention.

### BRIEF DESCRIPTION OF THE FIGURES

Various exemplary embodiments of the methods of this invention will be described in detail, with reference to the following figures, wherein:

FIGS. 1A-1E illustrate an assessment of retinal vasculature in control (FIG. 1A and FIG. 1C) and calcitriol treated (FIG. 1B and FIG. 1D) mice during oxygen-induced ischemic retinopathy (OIR). FIGS. 1A-1D are wholemounts showing immunohistochemical staining of retinal preparation in control vs. treated groups. FIG. 1E is a histogram illustrating that the effect of calcitriol on inhibiting neovascular growth in the retina is dose dependent. The difference in the degree of neovascularization between control and calcitriol-treated mice is significant (P<0.001, for all 3 groups). These experiments were repeated three times with similar results (FIGS. 1A and 1B: bar =500 μm; FIGS. 1C and 1D: bar =50 μm).

FIGS. 2A and 2B illustrate an assessment of vascular endothelial growth factor (VEGF) levels in eyes from control and calcitriol-treated mice. FIG. 2A is a Western blot of VEGF and β-catenin in control and calcitriol treated animals. FIG. 2B is a quantitiative assessment of relative band intensities of the Western blots shown in FIG. 2A.

FIGS. 3A and 3B illustrate the effects of calcitriol treatment on body weight. Body weights of control (FIG. 3A) and calcitriol-treated (5 μg/Kg), FIG. 3B) mice during oxygen-induced ischemic retinopathy were determined at P12 (before treatment) and at P17 (after treatment). Data in each bar are the mean values of body weights of 4 mice from 4 experiments; Bars; Mean±SD. There was significant weight gain in control mice from P12 to P17, while calcitriol-treated mice failed to gain weight (P<0.05). A similar lack of weight gain was observed in mice treated with the lower doses of calcitriol (0.5 and 2.5 μg/Kg).

FIG. 4 illustrates the effects of calcitriol on retinal endothelial cell proliferation. Retinal endothelial cells were incubated with different concentrations of calcitriol for 3 days. The degree of cell proliferation relative to the control treatment was determined using a nonradioactive cell proliferation assay as described below. Data are plotted as optical density (OD) vs. μM calcitriol dose. Calcitriol had no effect on endothelial cell proliferation at concentrations below 10 μM, and at 100 μM inhibited cell proliferation by 90%. These experiments were repeated four times with similar results.

FIGS. 5A-C show the effects of calcitriol on retinal EC migration and morphogenesis. Retinal EC migration in the presence of ethanol (control) or calcitriol (10 μM) was determined using wound migration (FIG. 5A and FIG. 5B) and transwell (FIG. 5C) assays as described below.

FIGS. 6A through 6D illustrate the effect of calcitriol on retinal endothelial cell capillary morphogenesis in Matrigel™. The ability of retinal endothelial cell to undergo capillary morphogenesis in the presence of solvent control (FIG. 6A) and calcitriol (10 μM) (FIG. 6B) in Matrigel™ was determined as described in above. Images were obtained after 18 h. Calcitriol diminished the ability of retinal endothelial cells to undergo capillary morphogenesis to the point that no capillaries are observed in FIG. 6B. This concentration of calcitriol had no significant effect on the proliferation of retinal endothelial cells. These experiments were repeated three times with similar results. Bar =40 μm. FIGS. 6C and 6D are higher magnifications (×100) of FIGS. 6A and 6B (×40) respectively.

### DETAILED DESCRIPTION OF THE EXEMPLARY EMBODIMENTS

Before the present methods are described, it is understood that this invention is not limited to the particular methodology, protocols, cell lines, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a compound” includes a plurality of such compounds and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

“Subject” means mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

As used herein, “administering” or “administration” includes any means for introducing [whatever] into the body, preferably into the systemic circulation. Examples include but are not limited to oral; buccal, sublingual, pulmonary, transdermal, transmucosal, as well as subcutaneous, intraperitoneal, intravenous, and intramuscular injection.

A “therapeutically effective amount” means an amount of a compound that, when administered to a subject for treating a disease, is sufficient to effect such treatment for the disease. The “therapeutically effective amount” will vary depending on the compound, the disease state being treated, the severity or the disease treated, the age and relative health of the subject, the route and form of administration, the judgment of the attending medical or veterinary practitioner, and other factors.

For purposes of the present invention, “treating” or “treatment” describes the management and care of a patient for the purpose of combating the disease, condition, or disorder. The terms embrace both preventative, i.e., prophylactic, and palliative treatment. Treating includes the administration of a compound of present invention to prevent the onset of the symptoms or complications, alleviating the symptoms or complications, or eliminating the disease, condition, or disorder.

The pharmaceutical preparations administerable by the invention can be prepared by known dissolving, mixing, granulating, or tablet-forming processes. For oral administration, the anti-infective compounds or their physiologically tolerated derivatives such as salts, esters, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

As used herein, “pharmaceutical composition” means therapeutically effective amounts of the anti-neovascular compound together with suitable diluents, preservatives, solubilizers, emulsifiers, and adjuvants, collectively “pharmaceutically-acceptable carriers.” As used herein, the terms “effective amount” and “therapeutically effective amount” refer to the quantity of active therapeutic agent sufficient to yield a desired therapeutic response without undue adverse side effects such as toxicity, irritation, or allergic response. The specific “effective amount” will, obviously, vary with such factors as the particular condition being treated, the physical condition of the subject, the type of animal being treated, the duration of the treatment, the nature of concurrent therapy (if any), and the specific formulations employed and the structure of the compounds or its derivatives. In this case, an amount would be deemed therapeutically effective if it resulted in one or more of the following: (a) ocular neovascular growth; and (b) the reversal or stabilization of occular neovascular growth. The optimum effective amounts can be readily determined by one of ordinary skill in the art using routine experimentation.

Pharmaceutical compositions are liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, milamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

The preparation of pharmaceutical compositions which contain an active component is well understood in the art. Such compositions may be prepared as aerosols delivered to the nasopharynx or as injectables, either as liquid solutions or suspensions; however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like or any combination thereof.

In addition, the composition can contain minor amounts of auxiliary substances such as wetting or emulsifying agents, pH buffering agents which enhance the effectiveness of the active ingredient.

The methods of administering an effective dose of the antiangiogenic composition of calcitriol according to the invention includes pharmaceutical preparations comprising the anti-angiogenic compound alone, or can further include a pharmaceutically acceptable carrier, and can be in solid or liquid form such as tablets, powders, capsules, pellets, solutions, suspensions, elixirs, emulsions, gels, creams, or suppositories, including rectal and urethral suppositories. Pharmaceutically acceptable carriers include gums, starches, sugars, cellulosic materials, and mixtures thereof. The pharmaceutical preparation containing the anti-infective compound can be administered to a subject by, for example, subcutaneous implantation of a pellet. In a further embodiment, a pellet provides for controlled release of anti-infective compound over a period of time. The preparation can also be administered by intravenous, intraarterial, or intramuscular injection of a liquid preparation oral administration of a liquid or solid preparation, or by topical application. Administration can also be accomplished by use of a rectal suppository or a urethral suppository.

Examples of suitable oily vehicles or solvents are vegetable or animal oils such as sunflower oil or fish-liver oil. Preparations can be effected both as dry and as wet granules. For parenteral administration (subcutaneous, intravenous, intraararterial, or intramuscular injection), the anti-neovascular compounds or its physiologically tolerated derivatives such as salts, esters, N-oxides, and the like are converted into a solution, suspension or expulsion, , if desired with the substances customary and suitable for this purpose for example, solubilizers or other auxiliaries. Examples are sterile liquids such as oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, preferred liquid carriers are oils with particularly exemplary embodiments being vegetable oil and the like.

Pharmaceutically acceptable carriers for controlled or sustained release compositions administerable according to the invention include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

It should be noted that, while the investigations described herein use a post-mortem histo-chemical examination of the mouse eye to determine the extent of visualization, there are a variety of methods known in the art to assess the visual acuity and/or pathology of the eye. For example, a “tangent screen” or “Goldmann perimeter” test effectively measures the size of the subjects visual field by moving an object or a light from the periphery of the field toward the center. Such examinations allow the identification of blind spots. Such examinations now also include computerized automated perimetry. Macular degeneration can be home-monitored by tests using an Amsler grid, which comprises a black card having a white grid with a white dot in its center. The subject then looks at the grid with one eye noting any distortion in the grid-lines. Unviewable areas indicate a blind spot while the appearance of wavy lines indicates there may be a vision problem.

More objective examinations of the eye may be made by a trained professional using various methods of ophthalmascopy. Ophthalmoscopy allows the physician to see into the eye using several types of instruments. Such instruments include, a direct ophthalmoscope, which is an instrument resembling a small flashlight with several lenses that can magnify the fundus or back of the eye by about 15 times; an indirect ophthalmoscope is an instrument resembling a miner's lamp that is worn about the head. While an indirect ophthalmoscope magnifies only 3 to 5 times it allows a wider angle of view with a better view of the fundus. A slit lamp is a binocular device having a narrow beam focused on the fundus and viewed through a microscope. This instrument provides greater magnification but a smaller field of view and is mainly used to view the center of the fundus and the optic nerve. Other, more quantitative methods include fluorescein angiography which allows clear visualization of the retinal blood vessels using a fluorescent dye visualized by a series of photographs.

The Invention:

Calcitriol (1α,25-dihydroxyvitamin D₃), the active hormonal form of vitamin D, has shown a protective role in a variety of cancers including prostate, breast, colon, and retinoblastoma. These effects are mediated through interaction of calcitriol with its receptor (vitamin D receptor, VDR), which arrests the cancerous cell cycle at the G_{O}-G₁ transition through up-regulation of cyclin dependent kinase inhibitors P21 and P27 (24, 25). Furthermore, it has been long recognized that vitamin D may have a preventive effect against certain cancers with this effect thought to be due to the induction of apoptosis. In retinoblastoma cells, this occurs through modulation of expression of bcl-2 family members (26).

Recent studies by Mantell and colleagues have demonstrated a potential antiangiogenic activity for calcitriol in some tumors (Mantell D J, et al., 1α,25-dihydroxyvitamin D₃ inhibits angiogenesis in vitro and in vivo. Circ. Res. 2000;87:214-220.). However, statistical analysis of the effect was not significant. In these studies, nude mice were injected with MCF-7 breast carcinoma cell that had been induced to overexpress VEGF and MDA-435S breast carcinoma cells. The results of these studies indicated that, while there appeared to be a decrease in vascularization of the tumors, there was no significant decrease in tumor size nor was there a difference in the proportion of MCF7 and MDA-435S cells present in the tumor. Thus, these studies do not provide a suitable physiological model for retinopathy. For example, studies on the efficacy of the use of vitamin D on arresting the growth of retinoblastomas show that vitamin D may be effective. However, this effect appears to be related to the presence of a high affinity receptor specific for calcitriol. These findings lead to the conclusion that inhibition by vitamin D is proportionate to the quantity and affinity of the vitamin D receptor of each particular cell type.

The effects of calcitriol on angiogenesis have been unclear. Calcitriol has been reported to decrease (Merke J, et al., Identification and regulation of 1,25-dihydroxyvitamin D₃ receptor activity and biosynthesis of 1,25-dihydroxyvitamin D₃: studies in culture bovine aortic endothelial cells and human dermal capillaries. J Clin Invest. 1989;83:1903-1915) or have no effect (Wang D S, et al., Anabolic effects of 1,25-dihydroxyvitamin D₃ on osteoblasts are enhanced by vascular endothelial growth factor produced by osteoblasts and by growth factors produced by endothelial cells. Endocrinology. 1997;138:2953-2962) on endothelial cell proliferation; to have no effect on capillary morphogenesis in vitro (Lansink M, et al., Effects of steroid hormones and retinoids on the formation of capillary-like tubular structures of human microvascular endothelial cells in fibrin matrices is related to urokinase expression. Blood. 1998;92:927-938.); and to inhibit angiogenesis in vivo (Oikawa T, et al. Inhibition of angiogenesis by vitamin D₃ analogues. Eur JPharmacol. 1990;178:247-250). Further, studies on the effect of vitamin D on retinoblastomas showed two contraindications to its use in ocular diseases.

The effects of calcitriol on retinal neovascularization, retinal endothelial cell proliferation, and capillary morphogenesis has not been previously addressed. In the investigations described herein the inventors show that calcitriol significantly blocked retinal neovascularization during OIR at doses shown to be effective in inhibition of retinoblastoma with minimal toxicity. The effects of calcitriol on inhibition of angiogenesis were independent of changes in VEGF expression. To the inventors knowledge these investigations show that calcitriol is one of the most potent inhibitors of angiogenesis in the OIR model.

The inventors' results with retinal endothelial cells are consistent with previous reports that the effects of calcitriol on endothelial cell proliferation were minimal. No significant inhibition of retinal endothelial cell proliferation was observation when calcitriol was used up to 10 μM. However, significant inhibition of retinal endothelial cell proliferation was observed when calcitriol was used at 50 μM and in higher concentrations. Surprisingly, calcitriol at 100 μM inhibited retinal endothelial cell proliferation by 90%. These inhibitory concentrations are much higher than those used in many studies that reported no or mild effects on endothelial cell proliferation. Therefore, inhibition of endothelial cell proliferation may require higher concentrations of calcitriol. However, calcitriol at 10 μM completely abolished the ability of retinal endothelial cell to undergo capillary morphogenesis. This concentration had no effect on retinal endothelial cell proliferation in short (3 days) or long (9 days) incubation with calcitriol. Thus, calcitriol is a potent inhibitor of endothelial cell capillary morphogenesis independent of its effect on endothelial cell proliferation. This is consistent with the in vivo data showing that retinal neovascularization was dramatically inhibited in the presence of chemotherapeutic doses of calcitriol.

Calcitriol

Pure crystalline calcitriol (provided by ILEX Oncology Inc., San Antonio, Tex.) was prepared for injection as previously described (Albert D M, et al., Vitamin D analogs, a new treatment for retinoblastoma: the first Ellsworth lecture. Ophthamic Genet. 2002; 23:137-156). Briefly, the crystalline calcitriol was dissolved in 100% ethanol for a stock solution of 1 mg/ml and stored in amber bottles under argon gas at −70° C. The stock solution was diluted in mineral oil to a concentrations of 0.0025, 0.0125 and 0.025 μg/0.1 ml. Each mouse in the treatment group received 0.0025, 0.0125 or 0.025 μg of calcitriol (approximately 0.5, 2.5 and 5 μg/Kg) per treatment. These doses were previously found (Albert D M, et al., Vitamin D analogs, a new treatment for retinoblastoma: the first Ellsworth lecture. Ophthamic Genet. 2002; 23:137-156, Sabet S J, et al., Antineoplastic effect and toxicity of 1,25-dihydroxy-16-ene-23-yne-vitamin D₃ in athymic mice with Y-79 human retinoblastoma tumors. Arch Ophthalmol. 1999;1 17:365-370) to be an effective dose with minimal toxicity. For in vitro studies, a stock solution of calcitriol in 100% ethanol (2 mM, 0.83 mg/ml) was prepared.

Mouse Model of Oxygen-Induced Ischemic Retinopathy

All experimental procedures involving animals were performed according to the ARVO Statement for the Use of Animals in Ophthalmic and Vision Research. The mouse oxygen-induced ischemic retinopathy (OIR) model (Smith L E, et al. Oxygen-induced retinopathy in the mouse. Invest. Ophthalmol. Vis. Sci. 1994; 35: 101-111) was used to evaluate the effects of calcitriol on retinal neovascularization. In this model, 7-day-old (P7) pups (8-10 pups) and their mother were placed in an airtight incubator and exposed to an atmosphere of 75±0.5% oxygen (hyperoxia) for 5 days. Incubator temperature was maintained at 23±2° C., and oxygen was continuously monitored with a PROOX model 110 oxygen controller (Reming Bioinstruments Co., Redfield, N.Y.). Mice were then brought to room air for 5 days. Maximum retinal neovascularization occurred from P12 to P17. To assess anti-angiogenic activity of calcitriol, half of the pups were injected intraperitoneally with 0.025 μg calcitriol in 0.1 ml mineral oil per day from P12 to P17. The other half of the littermates was injected with 0.1 ml of mineral oil. Generally one eye from each mouse was used for histochemical analysis and the other eye for histological evaluation as outlined below. These experiments were repeated at least 3 times for each dose.

Calcium Toxicity

A cytotoxic side effect of calcitriol treatment is loss of body weight due to hypercalcemia. The antineoplastic effect of calcitriol, however, is unrelated to either high serum calcium levels or calcium deposition in the tumors. In fact, the clinical usefulness of vitamin D is limited by the toxic effects associated with hypercalcemia. The inventors evaluated the body weights of mice injected with solvent control or calcitriol during OIR. The body weight of mice injected with solvent control from P12 to P17 was increased by 30%, while the body weight of mice injected with calcitriol was decreased by 20%. These are consistent with previous mouse studies and indicate a potential side effect of calcitriol treatment.

Visualization and Quantification of retinal Neovascularization

Vessel obliteration and retinal vascular pattern were analyzed using retinal wholemounts stained with anti-Collagen IV antibody as previously described (Wang S, et al. Thrombospondin-1-deficient mice exhibit increased vascular density during retinal vascular development and are less sensitive to hyperoxia-mediated vessel obliteration. Dev Dyn. 2003; 228:630-642., Wang S, et al. Attenuation of Retinal Vascular Development and Neovascularization during Oxygen-Induced Ischemic Retinopathy in Bcl-2 -/- Mice. Developmental Biol. 2005;279:205-219). Briefly, P17 mouse eyes were enucleated and briefly fixed in 4% paraformaldehyde in phosphate buffered saline (PBS) (10 min on ice). The paraformaldehyde fixed eyes were then fixed in 70% ethanol for at least 24 h at −20° C. Retinas were dissected in PBS and then washed with PBS 3 times, 10 min each. Following incubation in blocking buffer (50% fetal calf serum, 20% normal goat serum (NGS) in PBS) for 2h, the retinas were incubated with rabbit anti-mouse collagen IV (Chemicon, diluted 1:500 in PBS containing 20% fetal calf serum, 20% normal goat serum) at 4° C. overnight. Retinas were then washed 3 times with PBS, 10 min each, incubated with a secondary antibody Alexa 594-labeled goat-anti-rabbit (Invitrogen, Carlsbad, Calif.), at 1:500 dilution prepared in PBS containing 20% FCS, 20% NGS for 2 h at room temperature, washed 4 times with PBS (30 min each), and mounted on a slide with PBS/glycerol (2vol/1vol). Retinas were viewed by fluorescence microscopy, and images were captured in digital format using a Zeiss microscope (Carl Zeiss, Chester, Va.).

Quantification of retinal Neovascularization

Quantification of retinal neovascularization on P17 was performed by counting vascular cell nuclei anterior to the in limiting membrane (Wang S, et al., Attenuation of Retinal Vascular Development and Neovascularization during Oxygen-Induced Ischemic Retinopathy in Bcl-2 -/- Mice. Developmental Biol. 2005;279:205-219). Briefly, mice eyes were enucleated, fixed in formalin for 24 h, and embedded in paraffin. Serial sections (6 μm), each separated by at least 40 μm, were taken from around the region of the optic nerve. The hematoxylin- and PAS-stained sections were examined in masked fashion, by two independent observers without the knowledge of the samples identity, the presence of neovascular tufts projecting into the vitreous from the retina. The neovascular score was defined as the mean number of neovascular nuclei per section found in eight sections (four on each side of the optic nerve) per eye; generally four eyes from different mice per experiment were used.

Western Blot Analysis

Vascular endothelial growth factor (VEGF) levels were determined by Western blotting of whole eye extracts prepared from P15 mice during OIR (5 days of hyperoxia and 3 days of normoxia) when maximum levels of VEGF were expressed (Wang S, et al. Attenuation of Retinal Vascular Development and Neovascularization during Oxygen-Induced Ischemic Retinopathy in Bcl-2 -/- Mice. Developmental Biol. 2005;279:205-219). Mice were euthanized by CO₂ inhalation, then eyes from 2 or 3 mice dissected, homogenized in 0.2 ml of RIPA buffer, 10 mM HEPES pH 7.6, 142.5 mM KCl, 1% NP-40, and protease inhibitor cocktail, (Roche Applied Science, Indianapolis, Ind.), sonicated briefly, and incubated at 4° C. for 20 min. The resulting homogenates were then centrifuged at 16,000 ×g for 10 min at 4° C. to remove insoluble material. Supernatants were transferred to a clean tube, and protein concentrations were determined using the DC Protein Assay (Bio-Rad Laboratories, Hercules, Calif., Cat. No. 500-011 1). Approximately 20 μg of protein from the centrifuged homogenates were analyzed by SDS-PAGE (4-20% Tris-Glycin gel, Invitrogen, Carlsbad, Calif.) under reducing conditions and transferred to a nitrocellulose membrane. The blot was incubated with a rabbit polyclonal anti-mouse VEGF antibody (1:2000 dilution; PeproTech, Rock Hill, N.J.), washed, and developed using a goat anti-rabbit HRP-conjugated secondary antibody (1:5000; Jackson Immunoresearch Laboratories, West Grove, Pa.) and ECL system (Amersham Biosciences, Piscataway, N.J.). The same blot was also probed with a monoclonal antibody to β-catenin (1:3000; B D Transduction Laboratories, B D Biosciences, San Jose, Calif.) to verify equal protein loading in all lanes. For quantitative assessments, the band intensities relative to loading controls were determined by scanning the blots using the Molecular Dynamics Storm 860 Scanner and Image Quant Software (Amersham Biosciences, Piscataway, N.J.).

Toxic Effect Assessment

The side effects of calcitriol on the mouse body weights were determined. In the OIR studies, all pups had similar body weight prior to initiation of the experiment (P12) and after exposure to high oxygen (P17). None of the experimental animals died during these experiments.

Determination ofSerum Calcium Levels

Blood (0.2 ml) was collected from P17 mice treated with calcitriol or solvent control during OIR. The blood was allowed to clot at room temperature, centrifuged, the serum was transferred to a clean tube, and stored at −80° C. until needed for analysis. Serum samples were sent to Marshfield Clinic (Marshfield, Wis.) for total serum calcium analysis. The serum calcium level is reported as mg/dL.

Retinal EC Proliferation, Migration and Capillary Morphogenesis

Primary mouse retinal endothelial cell (REC) cultures were prepared and maintained as described previously (Su X, et al., Isolation and characterization of murine retinal endothelial cells. Mol Vision. 2003;9:171-178). Briefly, REC were isolated from wild type or transgenic-immortomouse by collagenase digestion of retina and affinity purification using magnetic beads coated with platelet/endothelial cell adhesion molecule-1 (anti-PECAM-1). The bound cells were plated on fibronectin-coated wells and expanded. The REC were characterized for expression and localization of endothelial cell markers by fluorescence-activated cell sorting (FACS) analysis and indirect immuno fluorescence staining. The ability of these cells to form capillary like networks was assessed on matrigel™. For cell proliferation assays, retinal endothelial cell (10,000) were plated in triplicate in 96-well plates and incubated overnight. On the following day, cells were fed with growth medium containing various concentrations of calcitriol or solvent control. Cells were allowed to grow for the indicated period of time and were fed every 3 days with fresh medium containing appropriate concentrations of calcitriol. The degree of proliferation was assessed using the nonradioactive cell proliferation assay (CellTiter 96® AQᵤₑₒᵤₛ; Promega, Madison, Wis.) as recommended by the supplier.

Retinal EC migration was determined using both wound migration and transwell assays. Confluent monolayers of retinal EC were wounded using a micropipette tip, rinsed with growth medium to remove detached cells, and incubated with growth medium containing calcitriol (10 μM) or ethanol (solvent control). Wound closure was monitored by phase microscopy, and digital images were obtained at different time points used for quantitative assessment of migration. For transwell migration, wells (8 μm pore size, 6.5 mm membrane; Costar) were coated with Matrigel™ (200 μg/ml) or fibronectin (2 μg/ml) in PBS on the bottom side at 4° C. overnight. The next day, inserts were rinsed with PBS, blocked in PBS containing 2% BSA for 1 h at room temperature, and washed with PBS. Cells were removed by trypsin-EDTA, counted, and resuspended at 1×10⁶ cells/ml in serum-free medium. Inserts were placed in 24-well dishes (Costar) containing 0.5 ml of serum-free medium and 0. 1 ml of cell suspension was then added to the top of the insert. Cells were allowed to migrate through the filter for 3 h in a tissue culture incubator. After incubation, the cells on the top of the filter were scraped off using a cotton swab; the membrane was then fixed in 4% paraformaldehyde and stained with hematoxylin and eosin. The inserts were then mounted on a slide cell side up, and the number of cells which migrated to the bottom of the filter was determined by counting 10 high power fields at X200 magnification.

The ability of the cultured retinal endothelial cells to form capillary like networks was assessed on Matrigel™ (B D Biosciences, San Jose, Calif.). The capillary morphogenesis assays in Matrigel™ were performed as previously described (Su X, et al., Isolation and characterization of murine retinal endothelial cells. Mol Vision. 2003;9:171-178; Rothermel T A, et al., Polyoma virus middle-T-transformed PECAM-1 deficient mouse brain endothelial cells proliferate rapidly in culture and form hemangiomas in mice. J Cell Physiol. 2005;202:230-239). Briefly, 0.5 ml of Matrigel™ was added to a cold 35 mm tissue culture plate and incubated at 37° C. for at least 30 min to allow the Matrigel™ to harden. Retinal endothelial cells were removed by trypsin-EDTA, resuspended at 1.5×10⁵ cells/ml in the growth medium containing calcitriol (10 μM) or solvent control, and incubated on ice for 15 min. Following incubation, 2 ml of cell suspension in the presence of calcitriol or solvent control was gently added to the Matrigel™-coated plates and incubated at 37° C. Cultures were monitored for 6-48 h, and images were captured in digital format after 18 h when maximum organization was observed. Longer incubation did not result in further organization of endothelial cells into tubular network. The capillary network formed by control cells began to fall apart at 24-48 h.

Statistical Analysis

Statistical differences between groups were evaluated with Student's unpaired t-test (two-tailed). Mean±standard deviation is shown. P values≦0.05 were considered significant.

### EXAMPLE 1

### Effects of Calcitriol on Retinal Neovascularization

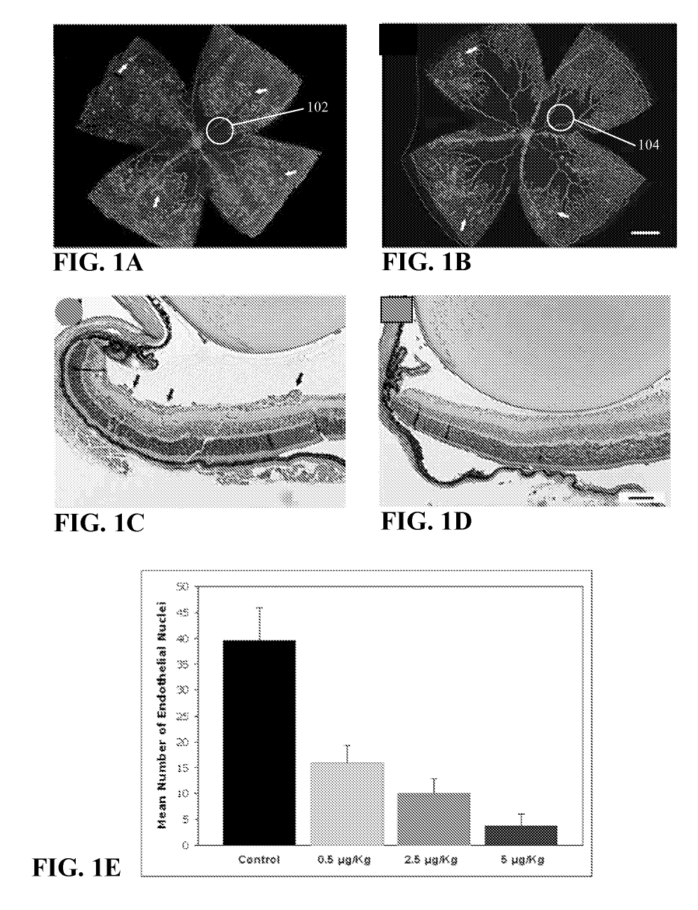

As described previously, the inventors, in performing experiments on the effects of various vitamin D analogs in treating retinoblastoma, included calcitriol as a control. Collagen IV immunohistochemical staining of the wholemount retinas was performed to visualize ischemia-induced retinal neovascularization. In this experiment, P7 mice were exposed to a cycle of hyperoxia and normoxia, and eyes were removed for appropriate analysis as described above. FIGS. 1A and 1B show retinal wholemounts in which the retinal vasculature was visualized by immunohistochemical staining using an anti-collagen IV antibody from P17 control and calcitriol-treated mice exposed to OIR, respectively. FIGS. 1C and 1D show hematoxylin-and periodic acid-Schiff (PAS)-stained cross sections prepared from P17 control and calcitriol-treated mice (0.5 μg/Kg, 2.5 μg/Kg and 5μg/Kg) exposed to OIR, respectively. Arrows show the new vessels growing into the vitreous compartment. The quantitative assessments of retinal neovascularization in eyes from P17 control and calcitriol-treated mice exposed to OIR are shown in FIG. 1E. Data in each bar are the mean values from 4 eyes of 4 mice; Bars; Mean±SD. The difference in the degree of neovascularization between control and calcitriol-treated mice is significant (P<0.001, for all 3 groups). These experiments were repeated three times with similar results (FIGS. 1A and 1B: bar=500 μm; FIGS. 1C and 1D: bar=50 μm).

As shown, calcitriol-treated and control P17 mice subjected to OIR demonstrated significant obliteration of the peripapillary retinal capillaries, whereas the larger, well-developed radial retinal vessels extending from the optic disc still existed in areas 102 and 104 shown in FIG. 1A and 1B. Retinas from P17 control mice exposed to OIR contained many neovascular tufts extending from the surface of the retina at the junction between the perfused and nonperfused retina (arrows, FIG. 1A). In contrast, retinas from P17 mice treated with calcitriol demonstrated markedly reduced neovascularization (arrows, FIG. 1B). These results show that retinal neovascularization in the treated mice was inhibited by greater than 90% at 5 μg/Kg of calcitriol as shown in FIG. 5E (P<0.001). A lower degree of inhibition was observed at lower doses of calcitriol. A 75% inhibition of neovascularization was observed at 2.5 μg/Kg of calcitriol, while 60% inhibition was observed at 0.5 μg/Kg of calcitriol. These data show that the inhibition of angiogenesis by calcitriol is a dose dependent response, highlighting the finding that the decrease in vascularization is an effect of calcitriol not a secondary response to increased serum calcium levels.

### EXAMPLE 2

### Inhibition of Retinal Neovascularization by Calcitriol

Retinas from P17 control mice subjected to OIR contained multiple neovascular tufts on their surface (arrows, FIG. 1C), with some extending into the vitreous. Retinas from mice treated with calcitriol showed significantly fewer preretinal neovascular tufts, P<0.001 (FIG. 1D). The neovascular tufts contained a significant number of neovascular nuclei anterior to the ILM as illustrated by the data shown in Table 1 and FIG. 1E. This data shows that in OIR mice treated with calcitriol at doses of 0.025 μg, retinal neovascularization was inhibited by greater than 90% when compared to the control mice.

TABLE 1
MEAN NUMBER OF ENDOTHELIAL NUCLEI (P < 0.001)

| | |
|---|---|
| CONTROL (n = 4) | 39.9 ± 6.4 (SD) |
| CALCITRIOL (n = 4) 0.025 μg (˜5 μg/Kg) | 3.8 ± 2.2 (SD) |

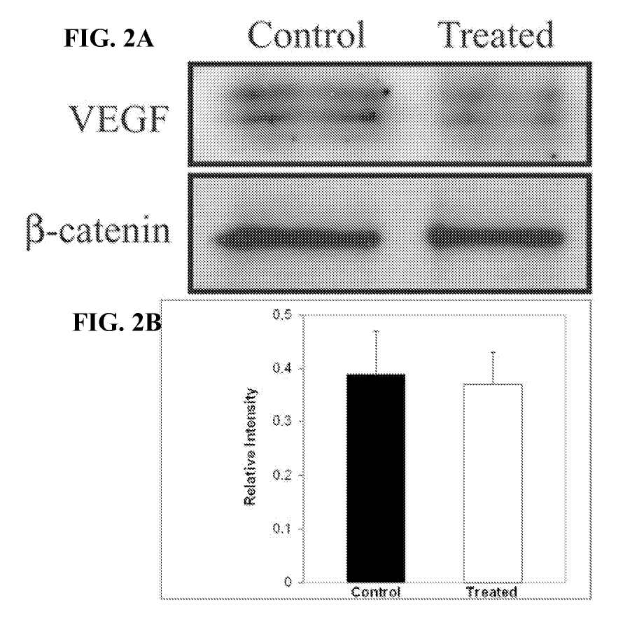

To determine whether the inability of retinas from calcitriol-treated mice to undergo neovascularization in response to ischemia was due to lack of VEGF expression Western blots were performed on the experimental animals. VEGF levels were examined in retinas from P15 control and calcitriol-treated mice during OIR (5 days of hyperoxia and 3 days of normoxia). It has been reported that VEGF expression is maximally induced at P15 during OIR (18). Briefly, eye extracts prepared from control and calcitriol-treated (5μg/Kg) P15 mice (5 days of hyperoxia and 3 days of normoxia) were analyzed by SDS-PAGE and Western blotting with β-catenin used for loading control (FIG. 2A). The quantitative assessments of relative band intensities are shown in (FIG. 2B). Data in each bar are the mean values of relative intensities of three experiments; Bars; Mean±SD. There was no significant difference in the relative amounts of VEGF expressed in control and calcitriol treated eyes (P<0.56). FIG. 2A shows a Western blot of protein prepared from whole eye extracts of control and calcitriol-treated P15 mice during OIR. The levels of VEGF expression and in eyes from control and calcitriol-treated mice during OIR were not significantly different P<0.56 (FIG. 2B). These data are provided in Table 2. The lack of any difference in VEGF expression between the treatment groups indicates that the effect of calcitriol on retinal neovascularization is not a result of differential VEGF expression but must result from some other mechanism. Further, the similarity in catenin expression indicates that the difference in retinal endothelial cell response is not due to an overall effect on protein expression but suggests that there is some more specific effect of calcitriol on neovascular growth.

TABLE 2
Relative Intensity of Expressed VEGF in Treatment Groups

| | |
|---|---|
| CONTROL (n = 3) | 0.39 ± 0.08 (SD) |
| CALCITRIOL (n = 3) 0.025 μg (˜5 μg/Kg) | 0.37 ± 0.06 (SD) |

### EXAMPLE 3

### Assessment of Side Effects of Calcitriol on the Body weight

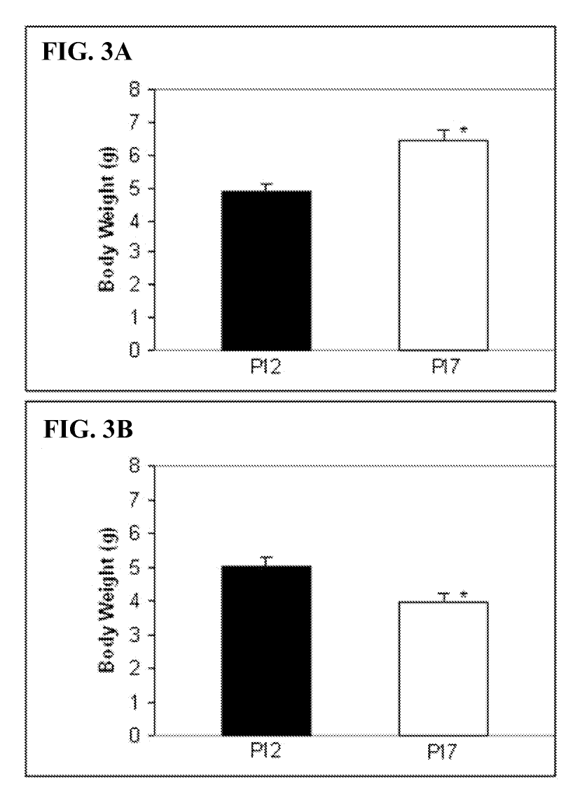

The body weights of experimental animals were determined at P12 and P17 after five days of injection with calcitriol or solvent control. In control mice, there was a significant increase in body weight of about 30% from P12 to P17 (FIG. 3A). In contrast, there was a significant decrease (20%) in the body weights of mice treated with calcitriol for 5 days (FIG. 3B; P<0.05). Thus, mice treated with calcitriol exhibit reduced bodyweights compared to control mice, a common side effect of calcitriol and hypercalcimia (Sabet S J, et al., Antineoplastic effect and toxicity of 1,25-dihydroxy-16-ene-23-yne-vitamin D₃ in athymic mice with Y-79 human retinoblastoma tumors. Arch Ophthalmol. 1999;1 17:365-370, Dawson D G, et al., Toxicity and dose-response studies of 1α-hydroxyvitamin D₂ in LHβ-Tag transgenic mice. Ophthalmology. 2003;1 10:835-839), this data is shown in Table 3.

TABLE 3
Change in Body Weight of Treatment Groups

| | P12 | P17 |
|---|---|---|
| Control (n = 4) | 4.85 ± 0.25 | 6.47 ± 0.29 |
| Calcitriol (n = 4) | 5.04 ± 0.23 | 3.93 ± 0.29 |

### EXAMPLE 4

### Calcitriol Inhibits Retinal Endothelial Cell Proliferation and Capillary Morphogenesis in Matrigel™

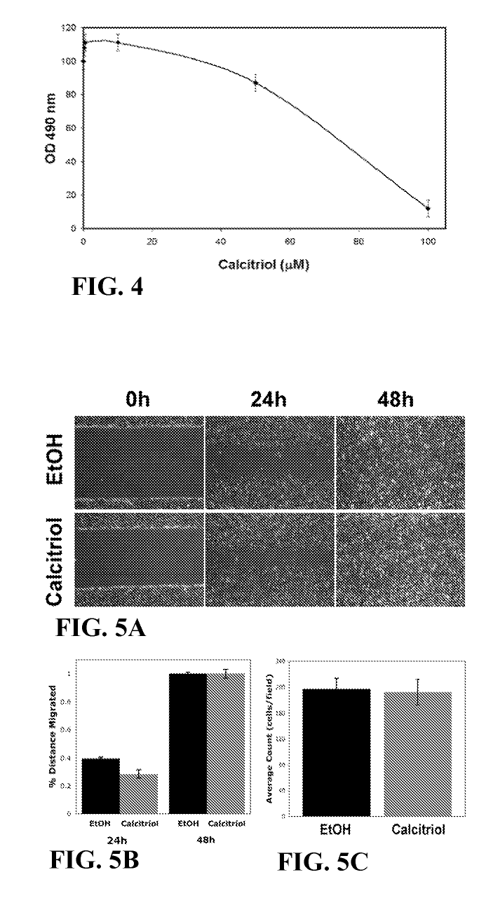

The effects of calcitriol on retinal endothelial cell proliferation have not been previously examined. Furthermore, the effects of calcitriol on proliferation of other types of endothelial cells have been contradictory. The inventors examined the effects of calcitriol on retinal endothelial cell proliferation, with both short-term (3 days) and long-term (9 days) incubation. Table 4 shows the proliferation of retinal endothelial cell incubated with (0 to 100 μM) concentrations of calcitriol relative to cells incubated with solvent control for 3 days at 37° C. Minimal toxicity was observed at lower concentrations of calcitriol (0-10 μM), and, in fact, low doses of calcitriol appear to result in an increase in cell proliferation when standardized to the control group. Significant toxicity was only observed at 50 μM calcitriol and higher. Calcitriol at 100 μM inhibited retinal endothelial cell proliferation by approximately 90%. Incubation of retinal endothelial cell with calcitriol (0-10 μM) for 9 days had minimal effects on their proliferation, similar to those observed after 3 days of exposure. This data is also illustrated in FIG. 4.

TABLE 4
Endothelial Cell Proliferation as
a Function of Calcitriol Treatment

| Calcitriol (μM) | Relative Survival |
|---|---|
| 0.00 | 100% |
| 0.25 | 108% |
| 0.50 | 111% |
| 10.00 | 111% |
| 50.00 | 87% |
| 100.00 | 12% |

### EXAMPLE 5

### Calcitriol Inhibits Retinal Endothelial Cell Migration

The effects of calcitriol on cell proliferation showing a biphasic response, the inventors then investigated the effects of calcitriol on retinal EC migration. FIGS. 5A shows the effect of- retinal EC migration in the presence of ethanol (control) or calcitriol (10 μM) as determined using wound migration and measured at 0, 24 and 48 hours after administration. The morphology of confluent monolayers of retinal EC wound closure was monitored by phase microscopy at different times post wounding and is shown in FIG. 5A. As shown in FIG. 5A there is little difference between the calcitriol and the control groups. FIG. 5B is a histogram illustrating the quantitative assessment of the two groups. A Student's unpaired t-test shows that the difference between the two groups is not significant.

FIG. 5C illustrate is a quantification of the transwell assay as described above. Briefly, wells were coated with Matrigel™ (200 μg/ml) or fibronectin (2 μg/ml) in PBS on the bottom side at 4° C. overnight. The next day inserts were rinsed with PBS, blocked in PBS containing 2%BSA for 1 h at room temperature and washed with PBS. Cells were removed by trypsin-EDTA, counted, and resuspended at 1×10⁶ cells/ml in serum-free medium. Inserts were placed in 24-well dishes (costar) containing 0.5 ml of serum-free medium, and 0.1 ml of cell suspension was then added to the top of the insert. Cells were allowed to migrate through the filter for 3 h in a tissue culture incubator. After incubation, the cells on the top of the filter were scraped off using a cotton swab. The membrane was fixed in 4% paraformaldehyde and stained with hematoxylin and eosin. The inserts were then mounted on a slide cell side up and the number of cells which migrated to the bottom of the filter was determined by counting 10 high power fields at ×200 magnification. Quantification of this assay (FIG. 5C) shows that there is no difference between the control group and the calcitriol treated group. Using the transwell assay, calcitriol had no significant effect on migration of retinal EC through the filter coated with Matrigel™ (FIG. 5C). However, calcitriol slightly enhanced retinal EC migration through filters coated with fibronectin compared to solvent control (not shown). Therefore, calcitriol at 10 μM had minimal effects on retinal EC migration in culture. For FIG. 5B, data in each bar are the mean values of percent distance migrated from three separate experiments; Bars, Mean±SD. For FIG. 5C, data in each bar are the mean values of cells migrated through the membrane in 10 high power fields of three separate experiments; Bars, Mean±SD. Note there is no significant difference in the degree of migration among control and calcitriol-treated cells (P<0.5)

### EXAMPLE 6

### Effects of Calcitriol on Retinal EC Capillary Morphogenesis in Matrigel™

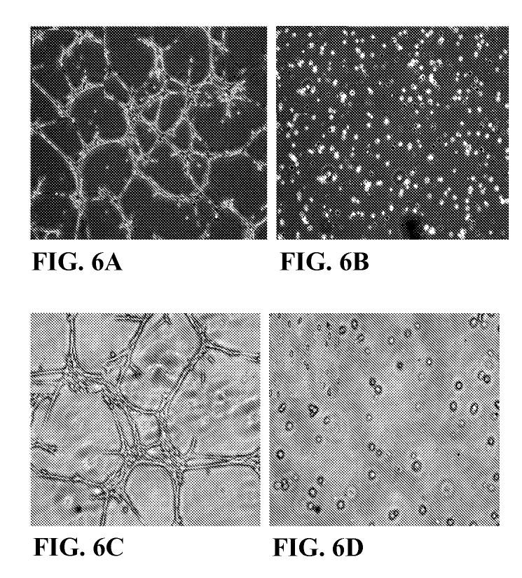

Because there was no decrease in the proliferation or migration of retinal EC cells treated with calcitriol, the inventors then investigated the ability of retinal EC cells to organize into capillary networks. Previous studies have shown that retinal endothelial cells, like many other types of endothelial cells, rapidly organize into capillary networks when plated in Matrigel™. In contrast to the proliferation assays, the morphogenesis assay reveals that, in vitro, even the presence of 10 μM calcitriol inhibits the ability of retinal EC cells to form capillary networks. FIGS. 6A and 6B are 40× magnifications of EC cells cultured on Matrigel™ without calcitriol (6A) and in the presence of 10 μM calcitriol. FIGS. 6C and 6D are the same preparations but at higher magnification (100×). As is shown, in the presence of 10 μM calcitriol capillary morphogenesis was completely inhibited. This concentration of calcitriol, as shown in Table 4, results in an increase in EC cell proliferation yet, as disclosed herein, results in a complete absence of capillary formation. This in vitro data is consistent with the in vivo data which shows the inhibition of retinal neovascularization by calcitriol as illustrated in FIGS. 1A and 1B and discussed above.

The inventors have shown that calcitriol, in vivo, inhibits retinal neovascularization by greater than 90% when compared to controls. Further, these effects were shown to be dose dependent such that, in vivo, inhibition of neovascular growth was induced at doses as low as 0.5 μg/Kg to 5 μg/Kg, doses which tended to stimulate EC cell proliferation in vivo. Thus, calcitriol in doses that have been found to be therapeutically effective can be used to inhibit neovascular growth, particularly in the retina. Therefore, systemic administration of calcitriol may be used as an efficacious treatment for non-neoplastic neovascular growth such as that exhibited in diabetic retinopathy, retinopathy of hypertension and wet macular degeneration.

Without being held to any particular theory, this data strongly suggests that calcitriol may exert its effects on cell growth and differentiation by, at least, two different mechanisms: one mechanism which results in an increase in cell proliferation at low doses and further has no effect on proliferation at high doses; and another mechanism which, while having no effect on proliferation, has a profound effect on capillary morphogenesis. While such mechanisms are not fully understood, these data may illustrate the effects of calcitriol that are independent of the vitamin D receptor or effects that are masked by calcium toxicity resulting from the high serum calcium concentration due to dosing animals with excessive amounts of calcitriol.

Further, it should be noted that, while in the studies described herein calcitriol was administered systemically by intraperitoneal injection, the route of calcitriol administration can be made by any effective means, as discussed previously. For example, it may be appreciated that, in some instances, the vitamin D compound of the invention is administered directly into the eye by means of drops, ophthalmic cream, a hydrogel or the like placed in the eye or under the eyelid. In addition, where the neovascular growth is superficial, such as, for example, a vascular birthmark, the vitamin D compound of the invention is administered topically as a cream or salve.

While this invention has been described in conjunction with the various exemplary embodiments outlined above, various alternatives, modifications, variations, improvements and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents of these exemplary embodiments.

What is claimed is:

1. A method of treating non-neoplastic neovascular growth in a subject in need thereof comprising administering an effective amount of calcitriol having a structure represented by Formula I or a salt or prodrug thereof wherein the non-neoplastic neovascular growth is decreased.

2. The method of claim 1, wherein the neovascular growth is in the eye.

3. The method according to claim 2, wherein the neovascular growth in the eye is due to a condition selected from the group consisting of diabetic retinopathy, hypertensive retinopathy, retinopathy of prematurity and macular degeneration.

4. The method of claim 1, wherein the calcitriol is administered systemically.

5. A method of inhibiting non-neoplastic neovascular growth, comprising administering an inhibiting amount of calcitriol having a structure represented by Formula I or a salt or prodrug thereof wherein non-neoplastic neovascular growth is inhibited.

6. The method of claim 5, wherein the non-neoplastic neovascular growth to be inhibited is in the eye.

7. The method according to claim 6, wherein the non-neoplastic neovascular growth to be inhibited is a manifestation of diabetic retinopathy, hypertensive retinopathy, retinopathy of prematurity or macular degeneration.

8. The method of claim 7, wherein the calcitriol is administered systemically.
